(19) United States
(12) Patent Application Publication
(10) Pub. No.: US 2009/0326642 A1
(43) Pub. Date: Dec. 31, 2009

(54) Implantable Medical Devices Fabricated From Radiopaque Polymers With High Fracture Toughness

(76) Inventor: Yunbing Wang, Sunnyvale, CA (US)

Correspondence Address: SQUIRE, SANDERS & DEMPSEY LLP, 1 MARITIME PLAZA, SUITE 300, SAN FRANCISCO, CA, 94111, US

(21) Appl. No.: 12/146,205

(22) Filed: Jun. 25, 2008

(51) Int. Cl. A61F 2/06

(52) U.S. Cl. 623/1.34

(57) ABSTRACT

Medical devices, such as stents, fabricated from a polymer including degradable polymer segments joined by di-urethane linkages with radiopaque functional groups chemically bonded to the polymer are disclosed.

### BACKGROUND OF THE INVENTION

1. Field of the Invention

This invention relates to implantable medical devices fabricated from biodegradable polymers and methods of fabricating such implantable medical devices. In particular, the invention relates to polymers with radiopaque groups grafted to the polymer.

2. Description of the State of the Art

This invention relates to radially expandable endoprosthesis, which are adapted to be implanted in a bodily lumen. An “endoprosthesis” corresponds to an artificial device that is placed inside the body. A “lumen” refers to a cavity of a tubular organ such as a blood vessel.

A stent is an example of such an endoprosthesis. Stents are generally cylindrically shaped devices, which function to hold open and sometimes expand a segment of a blood vessel or other anatomical lumen such as urinary tracts and bile ducts. Stents are often used in the treatment of atherosclerotic stenosis in blood vessels. “Stenosis” refers to a narrowing or constriction of the diameter of a bodily passage or orifice. In such treatments, stents reinforce body vessels and prevent restenosis following angioplasty in the vascular system. “Restenosis” refers to the reoccurrence of stenosis in a blood vessel or heart valve after it has been treated (as by balloon angioplasty, stenting, or valvuloplasty) with apparent success.

The treatment of a diseased site or lesion with a stent involves both delivery and deployment of the stent. “Delivery” refers to introducing and transporting the stent through a bodily lumen to a region, such as a lesion, in a vessel that requires treatment. “Deployment” corresponds to the expanding of the stent within the lumen at the treatment region. Delivery and deployment of a stent are accomplished by positioning the stent about one end of a catheter, inserting the end of the catheter through the skin into a bodily lumen, advancing the catheter in the bodily lumen to a desired treatment location, expanding the stent at the treatment location, and removing the catheter from the lumen. The stent may be visualized during delivery and deployment using X-Ray fluoroscopy if it contains radiopaque materials.

In the case of a balloon expandable stent, the stent is mounted about a balloon disposed on the catheter. Mounting the stent typically involves compressing or crimping the stent onto the balloon. The stent is then expanded by inflating the balloon. The balloon may then be deflated and the catheter withdrawn. In the case of a self-expanding stent, the stent may be secured to the catheter via a constraining member such as a retractable sheath or a sock. When the stent is in a desired bodily location, the sheath may be withdrawn which allows the stent to self-expand.

The stent must be able to satisfy a number of mechanical requirements. First, the stent must be capable of withstanding the structural loads, namely radial compressive forces, imposed on the stent as it supports the walls of a vessel. Therefore, a stent must possess adequate radial strength. Radial strength, which is the ability of a stent to resist radial compressive forces, is due to strength and rigidity around a circumferential direction of the stent. Radial strength and rigidity, therefore, may also be described as, hoop or circumferential strength and rigidity.

Once expanded, the stent must adequately maintain its size and shape throughout its service life despite the various forces that may come to bear on it, including the cyclic loading induced by the beating heart. For example, a radially directed force may tend to cause a stent to recoil inward. Generally, it is desirable to minimize recoil. In addition, the stent must possess sufficient flexibility to allow for crimping, expansion, and cyclic loading. Longitudinal flexibility is important to allow the stent to be maneuvered through a tortuous vascular path and to enable it to conform to a deployment site that may not be linear or may be subject to flexure. Finally, the stent must be biocompatible so as not to trigger any adverse vascular responses.

The structure of a stent is typically composed of scaffolding that includes a pattern or network of interconnecting structural elements often referred to in the art as struts or bar arms. The scaffolding can be formed from wires, tubes, or sheets of material rolled into a cylindrical shape. The scaffolding is designed so that the stent can be radially compressed (to allow crimping) and radially expanded (to allow deployment). A conventional stent is allowed to expand and contract through movement of individual structural elements of a pattern with respect to each other.

Additionally, a medicated stent may be fabricated by coating the surface of either a metallic or polymeric scaffolding with a polymeric carrier that includes an active or bioactive agent or drug. Polymeric scaffolding may also serve as a carrier of an active agent or drug.

Furthermore, it may be desirable for a stent to be biodegradable. In many treatment applications, the presence of a stent in a body may be necessary for a limited period of time until its intended function of, for example, maintaining vascular patency and/or drug delivery is accomplished. Therefore, stents fabricated from biodegradable, bioabsorbable, and/or bioerodible materials such as bioabsorbable polymers should be configured to completely erode only after the clinical need for them has ended.

Potential problems with polymeric stents include inadequate toughness and a degradation rate that is slower than is desirable for certain treatments. Another problem with polymeric stents is that polymers tend to be radiolucent, and thus cannot be visualized with X-ray fluoroscopy.

### SUMMARY OF THE INVENTION

### Brief Description of the Drawings

FIG. 1 depicts a stent.

FIG. 2 shows the half-life of PLLA and PGA homopolymers and copolymers implanted in rat tissue.

FIG. 3 depicts synthesis of PLLA diol.

FIG. 4 depicts the synthesis of a polyurethane polymer with PLLA segments.

FIG. 5 depicts synthesis of radiopaque polyurethane polymer from the polymer shown synthesized in FIG. 4 and triiodobenzoyl chloride.

FIG. 6 depicts synthesis of PLGA diol.

FIG. 7 depicts the synthesis of a polyurethane polymer with PLGA segments.

FIG. 8 depicts the synthesis of radiopaque polyurethane polymer from the polymer shown synthesized in FIG. 7 and triiodobenzoyl chloride.

### DETAILED DESCRIPTION OF THE INVENTION

As used herein, an “implantable medical device” includes, but is not limited to, self-expandable stents, balloon-expandable stents, stent-grafts, and generally tubular medical devices.

An implantable medical device can be designed for the localized delivery of a therapeutic agent. A medicated implantable medical device may be constructed by coating the device or substrate with a coating material containing a therapeutic agent. The substrate of the device may also contain a therapeutic agent.

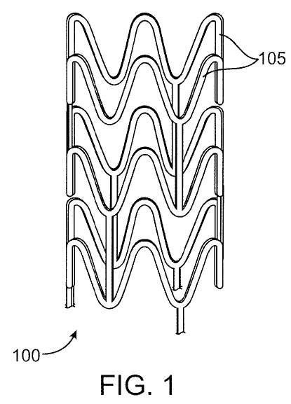

FIG. 1 depicts a view of a stent 100. In some embodiments, a stent may include a body, backbone, or scaffolding having a pattern or network of interconnecting structural elements 105. Stent 100 may be formed from a tube (not shown). The structural pattern of the device can be of virtually any design. The embodiments disclosed herein are not limited to stents or to the stent pattern illustrated in FIG. 1. The embodiments are easily applicable to other patterns and other devices. The variations in the structure of patterns are virtually unlimited. A stent such as stent 100 may be fabricated from a tube by forming a pattern with a technique such as laser cutting or chemical etching.

An implantable medical device can be made partially or completely from a biodegradable, bioabsorbable, or biostable polymer. A polymer for use in fabricating an implantable medical device can be biostable, bioabsorbable, biodegradable or bioerodable. Biostable refers to polymers that are not biodegradable. The terms biodegradable, bioabsorbable, and bioerodable are used interchangeably and refer to polymers that are capable of being completely degraded and/or eroded when exposed to bodily fluids such as blood and can be gradually resorbed, absorbed, and/or eliminated by the body. The processes of breaking down and absorption of the polymer can be caused by, for example, hydrolysis and metabolic processes.

Polymers generally do not possess sufficient radiographic density to be easily imaged by fluoroscopy. Since a device such as a stent also has specific mechanical requirements, it is important that rendering a polymeric stent sufficiently radiopaque for imaging does not significantly impact its mechanical behavior. One way to improve the imaging of polymeric materials is to incorporate radiopaque filler materials into a polymeric material to enhance radiographic density. However, such materials may adversely affect the mechanical properties of the polymer, such as strength and toughness.

An alternative to filler materials is to fabricate a stent from polymers having radiopaque dye or functional group covalently bonded to the polymer backbone and end groups. It is believed that the radiopaque density of the stent can be enhanced by the radiopaque functional groups without significantly impacting mechanical properties of the stent. Embodiments of the present invention include a stent fommed from a biodegradable polymer in which radiopaque functional groups are chemically bonded to the biodegradable polymer.

A radiopaque functional group can be any chemical group or species that can be bonded to a polymer backbone that increases or enhances the radiographic density of the polymer. Representative examples of radiopaque compounds from which radiopaque groups are derived include iodine, triiodobenzoyl bromide, triiodobenzoyl chloride, and triidobenzoic acid.

Additionally, some polymers that may be suitable for implantable medical devices such as stents have potential shortcomings. For example, some crystalline or semi-crystalline polymers may be selected primarily on the basis of strength and stiffness at physiological conditions so that the stent substrate or scaffolding can provide adequate support for a lumen. Physiological conditions refer to conditions within a human patient including, but not limited to, body temperature. Such polymers may be glassy or have a Tg above body temperature making them stiff and strong at body temperature which is approximately 37° C. A “glassy polymer” refers to a polymer having Tg above body temperature. One such shortcoming of such crystalline or semi-crystalline polymers is that their toughness is lower than desired, in particular, for use in stent applications. For example, polymers such as poly(L-lactide) (PLLA), polyglycolide (PGA), or copolymers thereof, poly(L-Lactide-co-glycolide) (PLGA), tend to be brittle under physiological conditions or conditions within a human body.

PGA, which is the simplest linear aliphatic polyester, degrades by hydrolysis of the easily accessible and hydrolytically unstable aliphatic-ester linkages. The degradation time, usually a few months, depends on multiple factors like molecular weight, degree of crystallinity, crystal morphology, and physico-chemical characteristics of the environment.

While structurally very similar to PGA, PLLA has some quite different chemical, mechanical and physical properties due to the presence of a pendant methyl group on the alpha carbon. This makes the PLLA a chiral molecule and the D, L, and DL isomers are possible. PLLA are semicrystalline polymers, while D,L-PLA is a completely amorphous material. The methyl group in PLLA makes the carbonyl of the ester link sterically less accessible to hydrolytic attack, which, depending on certain factors like the type of PLLA, its molecular weight, and its degree of crystallinity, makes the PLLA typically more hydrolytically stable than PGA when exposed to the same environment.

Another shortcoming of some biodegradable polymers is their degradation rate can be slower or faster than desired for certain stent treatments. For instance, the degradation rate may be too slow. As a result, the degradation time of a stent made from such a polymer can be longer than desired. Reducing degradation time allows further surgery or intervention, if necessary, on a treated vessel to occur sooner. Additionally decreasing degradation time helps cut down on the cases of late stent thrombosis, a condition in which clots form on the surface of the stent months or years after deployment. For example, a stent made from PLLA can have a degradation time of between about two and three years or longer. “Degradation time” refers to the time for a stent implanted in a vessel to completely absorb. “Degradation time” can also refer to the time for a stent to completely absorb under in vitro conditions. In some treatment situations, a degradation time of less than a year may be desirable, for example, between six and 12 months, or more narrowly, between eight and ten months.

The degradation of a hydrolytically degradable polymer follows a sequence including water penetration into the polymer followed by hydrolysis of bonds in the polymer. Thus, the degradation of a polymer can be influenced by its affinity for water and the diffusion rate of water through the polymer. A hydrophobic polymer has a low affinity for water which results in a relatively low water penetration. In addition, the diffusion rate of water through crystalline regions of a polymer is lower than amorphous regions. Thus, as either the affinity of a polymer for water decreases or the crystallinity increases, water penetration and water content of a polymer decreases, resulting in a slower degradation rate.

Various embodiments of the present invention include an implantable medical device, such as a stent, formed from a biodegradable polymer having radiopaque functional groups chemically bonded to the polymer. The radiopaque groups allow the stent to be visualized upon implantation within a body of a patient. In such embodiments, the radiopaque functional groups can be chemically bonded to or along the backbone of the polymer. Additionally, the radiopaque functional groups can also be bonded to end groups of the polymer.

In certain embodiments, an implantable medical device can be fabricated from a polymer including degradable polymer segments joined by di-urethane linkages. In such embodiments, the radiopaque functional groups may be chemically bonded to the di-urethane linkages along the backbone of the polymer. In some embodiments, the polymer can have the same type of polymer segments joined by the di-urethane linkages throughout the polymer chain. In alternative embodiments, the polymer can have different types of polymer segments. In each of these embodiments, the polymer segments can be homopolymers or copolymers.

In all such embodiments, one or more structural elements or struts of a stent can be fabricated from the polymer. In other such embodiments, the body, backbone, scaffolding, or substrate of a stent can be made from the polymer. The body, scaffolding, or substrate may be primarily responsible for providing mechanical support to walls of a bodily lumen once the stent is deployed within the bodily lumen. A stent body, backbone, scaffolding, or substrate can refer to a stent structure with an outer surface to which no coating or layer of material different from that of which the structure is manufactured. If the body is manufactured by a coating process, the stent body can refer to a state prior to application of additional coating layers of different material. By “outer surface” is meant any surface however spatially oriented that is in contact with bodily tissue or fluids. A stent body, backbone, scaffolding, or substrate can refer to a stent structure formed by laser cutting a pattern into a tube or a sheet that has been rolled into a cylindrical shape.

In some embodiments, a majority, substantially all, or all of the stent body, scaffolding, or substrate can be made from the polymer. Substantially all of the body can refer to greater than 90%, 95%, or greater than 99% of the body. In other embodiments, a device body can be coated with the radiopaque polyurethane polymer.

As described in more detail below, embodiments of the radiopaque polymer of the present invention can be made from a polyurethane polymer that includes biodegradable polymer segments joined by di-urethane linkages. The general form of such a polyurethane polymer is:

HO—(P₁—O(CO)NH—R—NH(CO)O-P₂)ₙ—OH

where P₁ is a first type of polymer segment, P₂ is a second type of polymer segment, NH(CO)O and O(CO)NH are urethane groups, and O(CO)NH—R—NH(CO)O is the di-urethane linkage. R is a functional group derived from a di-isocyanate, which is one of the reactants used in the formation of the polyurethane polymer. In general, P₁ or P₂, which can be the same or different, can have the form —PS—R_{I}—PS—, where PS is a polymer or copolymer segment. For example, P₁ or P₂ can have the form: -Mₘ-R_{I}-Mₘ-, where Mₘ is a polymer or oligomer segment of monomer units. R_{I} is a functional group derived from an alcohol used in the synthesis of the polymer or oligomer segment. The polymer or oligomer segments can be virtually any polymer or oligomer segment that can be incorporated into the synthesis scheme described below or a synthesis scheme known to one of ordinary skill in the art of polymer chemistry to synthesize the polyurethane polymer structure provided above. Exemplary polymer or oligomer segments include PLLA, PGA, PLGA, polycaprolactone (PCL), and poly(trimethylene carbonate) (PTMC), polydioxanone (PDO), poly(4-hydroxy butyrate) (PHB), poly(butylene succinate) (PBS), and block copolymers, as discussed in more detail below.

Also described in more detail below, the polyurethane polymer can be formed by reacting at least one type of polyol with a di-isocyanate (O═C═N—R—N═C═O). Various kinds of di-isocyanates can be used, such as aliphatic and aromatic di-isocyanates. Exemplary di-isocyanates include, but are not limited to, di-isocyanatomethane, di-isocyanatobutane, di-isocyanatoethane, di-isocyanatohexane, di-isocyanatocubane, lysine di-isocyanate, and di-isocyanatocyclohexane.

As described in more detail below, radiopaque groups can be grafted onto the backbone of the polyurethane polymer through reaction of a radiopaque compound with —NH groups (of the di-urethane linkages. In this reaction, a radiopaque functional group derived from the radiopaque compound is substituted for protons of the —NH groups. Additionally, a radiopaque functional group can be added to the end of an aliphatic polyester through an esterification reaction. In this reaction, a radiopaque group is substituted for a proton of a hydroxyl end group. The general form of a polyurethane polymer with radiopaque groups (Ro) at the ends and along the backbone is as follows:

R_{O}O—(P₁—O(CO)NR_{O}—R—NR_{O}(CO)O—P₂)ₙ—OR_{O}

In some embodiments, the P₁ and P₂ can be the same and can include semicrystalline polymer or oligomer segments having a Tg above body temperature. Such polymer or oligomer segments are referred to as “hard blocks.” Exemplary hard block polymers include PLLA, PGA and Polymandelide (PM). In certain embodiments, all or substantially all of the structural elements of stent can be formed from such a polyurethane polymer. An exemplary radiopaque polyurethane polymer includes R_{O}O-(PLLA′-O(CO)NR_{O}—R—NR_{O}(CO)O-PLLA′)ₙ-OR_{O}, where PLLA′ corresponds to -(LLA)ₘ-R_{I}-(LLA)ₘ-.

In further embodiments, the polymer or oligomer segments between di-urethane linkages groups can be a copolymer with functional groups selected to increase the degradation rate of the polymer. Such a functional group can have a greater affinity for water or be more hydrolytically active than other functional groups of the polymer. In a particular embodiment, the matrix copolymer can be poly(L-lactide-co-glycolide) (PLGA). The weight percent of the GA in the copolymer can be at least about 1%, 5%, 10%, 15%, 30%, 40%, 50%, or greater 50%. An exemplary radiopaque polyurethane polymer includes R_{O}-(PLGA-O(CO)NR_{O}—R—NR_{O} (CO)O-PLGA)ₙ-OR_{O}, where PLGA corresponds to -(GA-co-LLA)ₘ-R_{I}-(GA-co-LLA)ₘ-.

As the literature data in Table 1 shows, the degradation time of PLGA decreases from three to four years to less than six months as the GA content increases from 0 wt % to 50 wt %. PLGA is amorphous throughout a wide range of GA content due to the disruption in the regularity in the polymer chain by the addition of another monomer. The decrease in degradation time is due both to the decreased crystallinity and increased hydrolytic activity of the GA.

TABLE 1
Degradation time of PLGA at different monomer compositions.

| | Degradation Time |
|---|---|
| Polymer | (months)^{a} |
| PGA | 6-12^{1,2} |
| 85/15 PLGA | 5-6¹ |
| 75/25 PLGA | 4-5¹ |
| 65/35 PLGA | 3-4¹ |
| 50/50 PLGA | 1-2¹ |

¹Medical Plastics and Biomaterials Magazine, March 1998.
²Medical Device Manufacturing & Technology 2005.

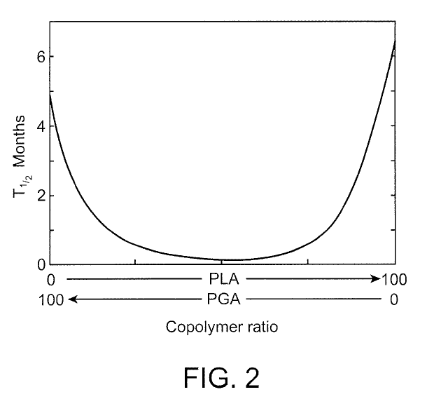

Furthermore, as shown by FIG. 2, the linear relationship between the copolymer composition and the mechanical and degradation properties of the materials is not linear. FIG. 2 shows the half-life of PLLA and PGA homopolymers and copolymers implanted in rat tissue. Journal of Biomedical Materials Research, 11:711, 1977. The half-life in FIG. 2 refers to the amount of time for the modulus of a polymer to decrease by one half due to degradation of the polymer. For example, a copolymer of 50% GA and 50% LLA degrades faster than either homopolymer.

In some embodiments, a stent scaffolding can be formed from a polyurethane polymer having PLGA segments or oligomers to decrease in degradation time. The decrease can be due to the GA content of the segments or oligomers. In certain exemplary embodiments, the weight percent of GA content can be adjusted so that the degradation time of a stent scaffolding can be less than 24 months, 16 months, 12 months, or more narrowly, less than 12 months.

In additional embodiments, an implantable medical device, such as a stent, can be fabricated at least in part from a radiopaque polyurethane polymer having polymer segments or oligomers with soft blocks. Soft blocks refer to polymers having a Tg below body temperature which tends to be elastomeric at physiological conditions. An “elastomeric” or “rubbery” polymer refers to a polymer which can resist and recover from deformation produced by force, as in natural rubber. In one embodiment, elastomers or rubbery polymers can be stretched repeatedly to at least twice their original length and, immediately upon release of the stress, return with force to their approximate original length. Elastomeric polymers tend to have a percent elongation at break larger than lower toughness polymers, such as brittle polymers. Biodegradable soft block polymers include, but are not limited to, polycaprolactone (PCL) and poly(trimethylene carbonate) (PTMC), polydioxanone (PDO), poly(4-hydroxy butyrate) (PHB), and poly(butylene succinate) (PBS). An exemplary radiopaque polyurethane polymer having oft block polymer segments includes R-(PDO′—O(CO)NR_{O}—R—NR_{O}(CO)O—PDO′)ₙ—OR_{O}, where PDO′ corresponds to -DOₘ-R_{I}-DOₘ-.

In further embodiments, an implantable medical device can be fabricated at least in part of a radiopaque polyurethane polymer having hard block and soft block polymer segments joined by di-urethane linkages. The soft block segments can increase the toughness of the polyurethane polymer. Additionally, a soft block polymer segment with a higher degradation rate than the hard block polymer segment can decrease the degradation time of a device. An exemplary radiopaque polyurethane polymer with hard and soft blocks can include: R_{O}O-(PLLA′-O(CO)NR_{O}—R—NR_{O}(CO)O-PDO′)ₙ—OR_{O}. The toughness and degradation rate can be tuned by the weight percent of the soft blocks in the polymer. In some embodiments, the molecular weight of the soft blocks can be between about 0.2 kg/mol to 2 kg/mol, 2 kg/mol to 20 kg/mol, or greater than 20 kg/mol.

In other embodiments, the soft block segment can be a copolymer. Exemplary copolymer soft blocks can be P(GA-co-CL), (GA-co-TMC), P(DO-co-CL) or P(DO-co-TMC). random or alternating copolymer segments. An exemplary block copolymer can include HO—(PLLA′-O(CO)NH—R—NH(CO)O—P(GA-co-CL)′)ₙ—OH.

In further embodiments, an implantable medical device can be fabricated at least in part of a radiopaque polyurethane polymer having degradable block copolymer segments joined by di-urethane linkages, the block copolymer segments including hard blocks and soft blocks. In some embodiments, the soft blocks can be homopolymer segments, for example, PCL, PDO, or PTMC. In other embodiments, the soft blocks can be random or alternating copolymer segments. For example, the random or alternating copolymer segment can include GA to increase the degradation rate of the segment and a soft block functional group, such as, CL, DO, or TMC. Thus, exemplary block copolymer segments can include PLLA-b-PCL, PLLA-b-PDO, PLLA-b-P(GA-co-CL), PLGA-b-PCL, PLGA-b-P(GA-co-CL).

In these embodiments, the soft blocks can form a dispersed elastomeric phase within a glassy polymeric matrix that includes the hard blocks. The presence of the dispersed elastomeric phase tends to increase the toughness of the radiopaque polymer and the implantable medical device. It is believed that when a device is placed under stress, a dispersed elastomeric phase tends to absorb energy when a fracture starts to propagate through a structural element. Crack propagation through a glassy polymer matrix may then be reduced or inhibited. As a result, fracture toughness of a polymer material and thus structural elements of a device fabricated from the polymer material is increased. Thus, brittle polymers can be toughened by incorporating into their microstructure higher toughness components that can act as “stress concentrators”, due to their lower tensile modulus.

The dispersed elastomeric phase can have an elongation at break greater than the polymeric matrix and a modulus less than the polymeric matrix. In exemplary embodiments, the dispersed phase can have an elongation at break at least 2, 4, 10, 100 times greater or more than 100 times greater than the polymer matrix. In other exemplary embodiments, the dispersed phase can have a modulus less than 20%, 50%, or less than 80% of the polymer matrix.

As indicated above, degradation rate of the block copolymer segment can increased by a GA functional group in the soft block. The weight fraction of GA can be varied between 0 and 50 wt %, for example, to vary the degradation rate of the block copolymer. For instance, variation of the GA content can vary the degradation time of a device body made from the radiopaque polymer from three years to six months.

As discussed above, the general form of a radiopaque polyurethane polymer is:

R_{O}O—(P₁—O(CO)NRO—R—NR_{O}(CO)O—P₂)ₙ—OR_{O}

For the radiopaque polyurethane polymer with the block copolymer, P₁ or P₂ have the form: —BC—R_{I}—BC—, wherein BC is the block copolymer segment having the hard and soft blocks. For example, a exemplary polymers include:

R_{O}O-(PLLA-b-PCL′-O(CO)NR_{O}—R—NR_{O}(CO)O-PCL-b-PLLA′)ₙ-OR_{O}

and

R_{O}O-(PLLA-b-P(GA-co-CL)′—O(CO)NR_{O}—R—NR_{O}(CO)O—P(GA-co-CL)-b-PLLA′)ₙ-OR_{O}

where PLLA-b-P(GA-co-CL)′ corresponds to PLLA-b-P(GA-co-CL)-R_{I}-P(GA-co-CL)-b-PLLA. The molecular weight of hard block segments such as PLLA or PLGA can be between 200 and 50,000 g/mol. The molecular weight of the soft block segments such as P(GA-co-CL) can be between 200 and 20,000 g/mol.

Embodiments of the radiopaque polyurethane polymers described herein can be formed by solution-based polymerization. In other embodiments, such polymers can be formed through melt phase polymerization. In solution-based polymerization, in each of the synthesis steps the reactive components involved in the polymerization reaction are dissolved in a solvent. In melt phase polymerization, the reactions can take place, for example, in a mixing device such as extruder.

A radiopaque polyurethane polymer can be formed in three steps. In the first step, biodegradable polymer chains with —OH or —NH₂ end groups are prepared through polymerization of monomer units. The chain length can be controlled by changing the ratio of monomer units to initiator, the reaction time, and reaction temperature. In the second step, the polymer chains are connected together through the reaction between their end groups and di-isocyanate. The di-isocyanate functional groups in the polymer that is formed has reactive —NH groups. In the third step, radiopaque groups are grafted onto the backbone of the polymer through reaction of iodine-substituted compounds with —NH groups.

As an illustration, a first step involving the formation of a PLLA diol is as follows:

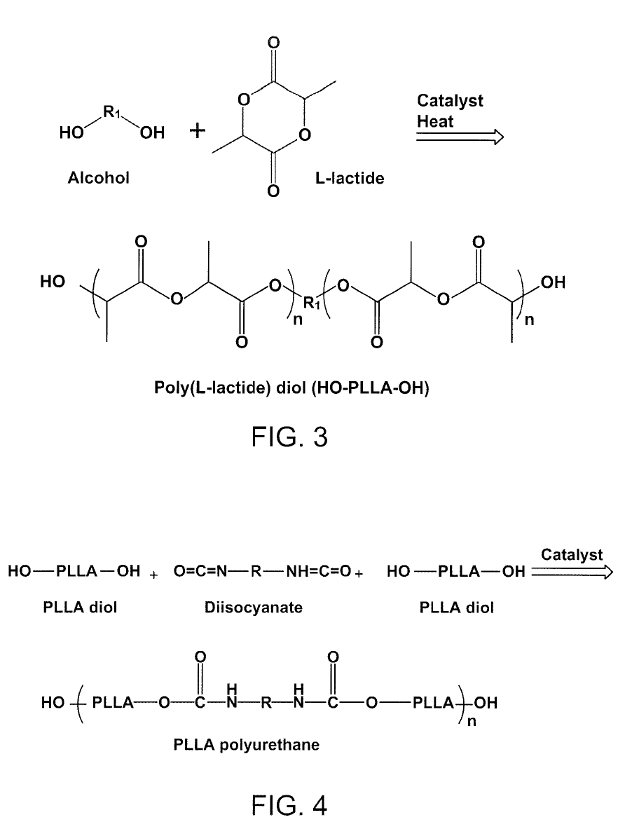

HO—R_{I}—OH+LLA→HO-(LLA)ₘ-R_{I}-(LLA)ₘ-OH (I)

where HO—R_{I}-OH is a diol initiator with a functional group R_{I}, HO-(LLA)ₘ-R_{I}-(LLA)ₘ-OH is a PLLA diol which will be written as HO-PLLA′-OH. An exemplary diol initiator is ethylene glycol. The reaction occurs in at an elevated temperature (normally greater than 80° C.) in a suitable solvent and in the presence of a suitable catalyst such a stannous octoate. The reaction is further illustrated in FIG. 3.

The PLLA diols react in solution with a di-isocyanate in the presence of a catalyst to form a polyurethane polymer as follows:

HO-PLLA′-OH+HO-PLLA′-OH+O═C═N—R—N═C═O→HO-(PLLA′-O(CO)NH—R—NH(CO)O-PLLA′)ₙ-OH (II)

The reaction is further illustrated in FIG. 4. The reaction occurs at room temperature (20-30° C.) or at an elevated temperature (greater than room temperature) in a suitable solvent and in the presence of a suitable catalyst such as stannous octoate.

Iodine-substituted compounds such as triiodobenzoyl chloride, triiodobenzoyl bromide, or triiodobenzozic acid can then be added to a solution containing synthesized PLLA diol polymer. The triiodobenzoyl chloride reacts with the PLLA diol polymer as follows:

HO-(PLLA′-O(CO)NH—R—NH(CO)O-PLLA′)ₙ-OH+C₆H₂I₃CO CL→R_{O}O-(PLLA′-O(CO)NR_{O}—R—NR_{O}(CO)O-PLLA′)ₙ-OR_{O} (III)

where R_{O} is C₆H₂I₃CO—.

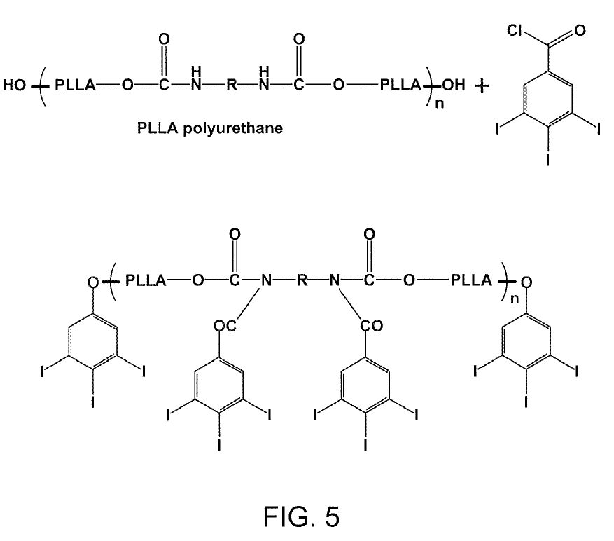

The triiodobenzoyl group is substituted for the protons of the end groups and the —NH groups. The reaction is further illustrated in FIG. 5.

Iodine-substituted compounds such as triiodobenzoyl chloride or triidobenzoic acid can then be added to a solution containing synthesized polyurethane block copolymer. Following synthesis of the radiopaque polyurethane polymer, the polymer is precipitated from the reaction solution by pouring the solution into a nonsolvent of the polymer.

As a further illustration, a first step involving the formation of a PLGA diol is as follows:

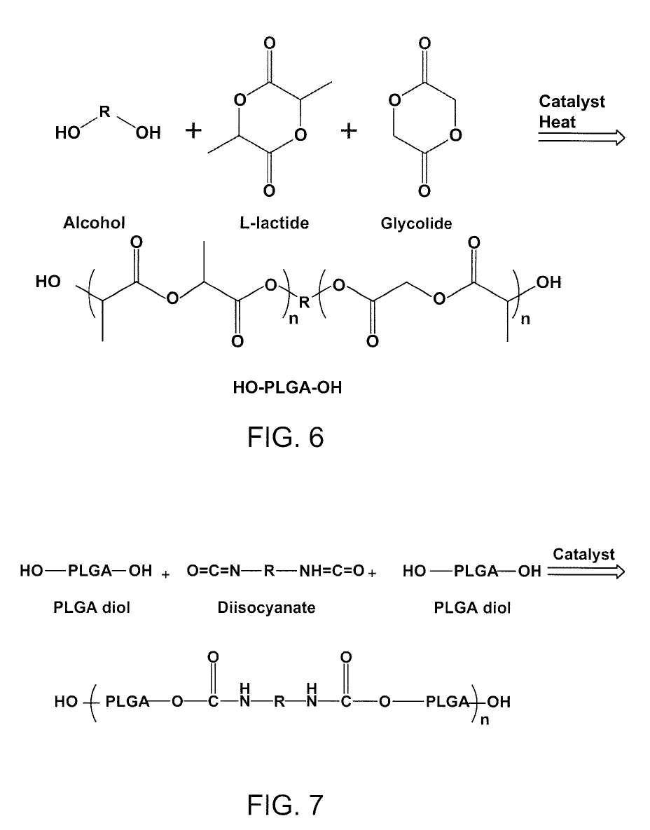

HO—R_{I}—OH+GA+LLA→HO—P(GA-co-LLA)-R_{I}—P(GA-co-LLA)-OH (IV)

where HO-P(GA-co-LLA)-R_{I}-P(GA-co-LLA)-OH is a PLGA diol which will be written as HO-PLGA′-OH. The reaction occurs in at an elevated temperature (normally greater than 80° C.) in a suitable solvent and in the presence of a suitable catalyst such a stannous octoate. The reaction is further illustrate in FIG. 6.

The PLGA diol reacts in solution with a di-isocyanate in the presence of a catalyst to form a polyurethane polymer as follows:

HO-PLGA′-OH+HO-PLGA′-OH+O═C═N—R—N═C═O→HO-(PLGA′-O(CO)NH—R—NH(CO)O-PLGA′)ₙ-OH (V)

The reaction is further illustrated in FIG. 7. The reaction occurs at room temperature (20-30° C.) or at an elevated temperature (greater than room temperature) in a suitable solvent and in the presence of a suitable catalyst such as stannous octoate.

Iodine or an iodine-substituted compounds such as triiodobenzoyl chloride can then be added to a solution containing the synthesized polyurethane block copolymer:

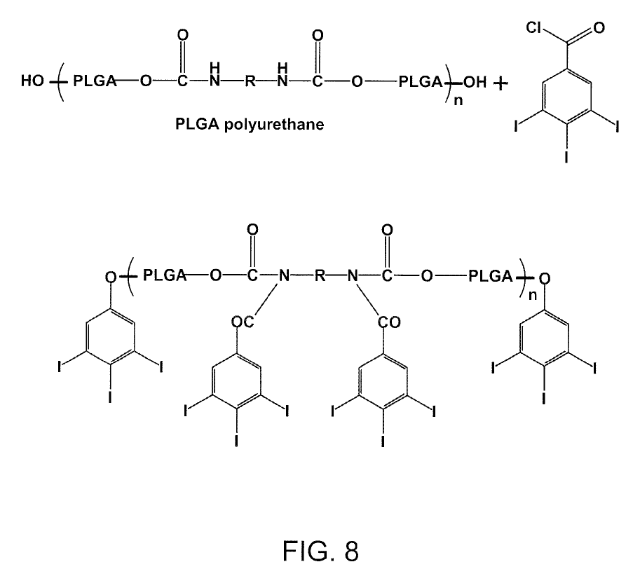

HO-(PLGA′-O(CO)NH—R—NH(CO)O-PLGA′)-OH+C₆H₂I₃CO CL→R_{O}O-(PLGA′-O(CO)NR_{O}—R—NR_{O}(CO)O-PLGA′)-OR_{O} (VI)

where R_{O} is C₆H₂I₃ CO—. The triiodobenzoyl group is substituted for the protons of the end groups and the —NH groups. The reaction is further illustrated in FIG. 8. Following synthesis of the radiopaque polyurethane polymer, the polymer is precipitated from the reaction solution by pouring the solution into a nonsolvent of the polymer.

As a further illustration, the radiopaque polyurethane polymer with soft block and hard block segments joined by a di-urethane linkage can be prepared in three steps. In the first step, hard block diol and soft block diol are prepared (e.g., PLLA diol and PCL diol) with —OH or —NH₂ end groups through the polymerization of the respective blocks segments (e.g., LLA and CL). In the second step, the hard block segments and soft block segments are connected together through the reaction of their end groups with isocyanate to form the polyurethane polymer. The polyurethane polymer chains contain —NH reactive functional groups in their backbone. In the third step, radiopaque groups are grafted onto the polyurethane polymer backbone through the reaction of iodine-substituted compounds.

With respect to the first step, the formation of the diols is mentioned above, e.g., HO-PLLA′-OH and HO-PCL′-OH. The hard and soft block diols react in solution with a di-isocyanate in the presence of a catalyst to form a polyurethane polymer as follows:

HO-PLLA′-OH+HO-PCL′-OH+O═C═N—R—N═C═O→HO-(PLLA′-O(CO)NH—R—NH(CO)O-PCL′)ₙ-OH (VII)

The reaction occurs at room temperature (20-30° C.) or at an elevated temperature (greater than room temperature) in a suitable solvent and in the presence of a suitable catalyst such as stannous octoate. The toughness the polymers can be adjusted by using different amount of PCL diol during polyurethane synthesis.

Iodine or an iodine-substituted compounds such as triiodobenzoyl chloride can then be added to a solution containing the synthesized polyurethane block copolymer:

HO-(PLLA′-O(CO)NH—R—NH(CO)O-PCL′)ₙ-OH+C₆H₂I₃ CO CL→R_{O}O-(PLLA′-O(CO)NR_{O}—R—NR_{O}(CO)O-PCL′)ₙ-OR_{O} (VIII)

where R_{O} is C₆H₂I₃ CO—.

As a further illustration, the radiopaque polyurethane polymer with a block copolymer with hard and soft blocks joined by di-urethane linkages can be prepared in three steps. In the first step, the block copolymer segments with hard and soft blocks (e.g., PLLA and PCL with —OH or —NH₂ end groups are prepared through the polymerization of the soft blocks segments (e.g., CL). After formation of the soft block segments, hard block monomers (e.g., LLA) are added to the reaction solution and polymerized to form the hard block of the block copolymer. In the second step, the block copolymer segments are connected together through the reaction of their end groups with isocyanate to form the polyurethane polymer. The polyurethane polymer chains contain —NH reactive functional groups in their backbone. In the third step, radiopaque groups are grafted onto the polyurethane polymer backbone through the reaction of iodine-substituted compounds.

The first step is illustrated as follows:

HO—R_{I}—OH+CL HO-PCL-R_{I}-PCL-OH (IX)

HO-PCL-R_{I}-PCL-OH+LLA→HO-PLLA-b-PCL-R_{I}-PCL-b-PLLA-OH (X)

where HO-PLLA-b-PCL-R_{I}-PCL-b-PLLA-OH is a diol which will be written as HO-PLLA-b-PCL′-OH. The reaction occurs at an elevated temperature (normally greater than 80° C.) in a suitable solvent and in the presence of a suitable catalyst such as stannous octoate.

The block copolymer diol reacts in solution with a di-isocyanate in the presence of a catalyst to form a polyurethane polymer as follows:

HO-PLLA-b-PCL′-OH+O═C═N—R—N═C═O→HO-(PLLA-b-PCL′-O(CO)NH—R—NH(CO)O-PCL-b-PLLA′)ₙ-OH (XI)

The reaction occurs at room temperature (20-30° C.) or at an elevated temperature (greater than room temperature) in a suitable solvent and in the presence of a suitable catalyst such as stannous octoate. Iodine or an iodine-substituted compounds such as triiodobenzoyl chloride can then be added to a solution containing the synthesized polyurethane block copolymer:

HO-(PLLA-b-PCL′-O(CO)NH—R—NH(CO)O-PCL-b-PLLA′)ₙ-OH+C₆H₂I₃CO CL→R_{O}O-(PLLA-b-PCL′-O(CO)NR_{O}—R—NR_{O}(CO)O-PCL-b-PLLA′)ₙ-OR_{O} (XII)

where Ro is C₆H₂I₃ CO—.

Solvents used in the solution polymerization synthesis of the polymer include, but are not limited to, chloroform, THF, dioxane, toluene, xylene, and cyclohexane. Initiators include, but are not limited to, ethylene glycol, propylene glycol, butane diol, and polyethylene glycol. Catalysts used to facilitate the synthesis of the polymer include, but are not limited to, stannous octoate, and stannous trifluoromethane sulfonate.

The intensity of transmitted X-rays, and thus the visibility of a radiopaque material using an imaging technique, depends at least of the weight fraction of radiopaque elements such as iodine in the material. In some embodiments, the fraction of iodine in the radiopaque polymer or stent formed from the polymer can be greater 1-5 wt %, 5-10 wt %, 10-15 wt %, 15-20 wt %, 20-30 wt %, or greater than 30 wt %.

The general form of the radiopaque polymer from above is:

R_{O}O—(P₁-O(CO)NR_{O}—R—NR_{O}(CO)O-P₂)ₙ—OR_{O}

The fraction of iodine in the polymer depends at least on the number of repeat units “n”, the molecular weight of P₁ and P₂, the number of iodine atoms in R_{O}.

The weight fraction of iodine in the polymer is:

MW of all I atoms in polymer/[MW of all I atoms in polymer+MW of all other atoms in polymer]

Specifically, this can be expressed as:

2×N_{I}×MW_{I}(n+1)/[2×N₁×MW_{I}(n+1)+2MW_{Rf}(n+1)+2×MW_{O}+n(MW_{P1}+MW_{P2}+MW_{N—R—N})]

where N_{I} is the number of iodine atoms in the radiopaque group, for example, triiodobenzoyl chloride and triidobenzoic acid each have three iodione atoms per molecule. Rf is functional group bonded to the iodine atom in R_{O}. The molecular properties of the radiopaque polymer can be tailored to obtain a selected or threshold weight fraction, T. From the above relationship, the number of repeat units, n, and the molecular weight of P₁ and P₂ have the greatest influence on the weight fraction of iodine.

As an illustration, once a value of n is selected, the maximum value of the combined molecular weight of P₁ and P₂ can be calculated to obtain the threshold weight fraction T. Alternatively, the combined molecular weight of P₁ and P₂ can be selected to allow calculation of the maximum value of n to obtain the threshold weight fraction T.

In an exemplary embodiment, T=0.15 and n=1, R=—CH₂CH₂—, R_{O} is a triiodobenzoyl group. In this example, the combined molecular weight of P₁ and P₂ must be less than or equal to about 9000 g/mole to provide the desired threshold weight fraction of iodine.

In exemplary embodiments, the molecular weight of P₁ or P₂ in the polymer can be 200-2000 g/mol, or in some instances greater than 2000 g/mol. In additional exemplary embodiments, the n can be 1-5, 5-10, or greater than 10.

The molecular weight of P₁ and P₂ can be controlled by the type and amount of diol initiator in, for example, reactions I and IV. The number of repeat units, n, in the polymer can be controlled by the ratio of diol to di-isocyanate in the reaction mixture that forms the polyurethane polymer, for instance, in reactions II and V. It is desirable to have an excess of diol so that the end groups of the formed polymer are hydroxyl groups, however, polymers with di-urethane end groups are within the scope of the present invention. In general, when the diol is in excess, as the ratio of diol to di-isocyanate increases the molecular weight of the formed polymer in reactions II and V tends to decrease. In exemplary embodiments, the ratio of diol to di-isocyanate can be between 1.02 and 1.08. Other embodiments can include ratios outside of this range.

In various embodiments, a stent or stent body or scaffolding can be formed in whole or in part of the radiopaque polymer. In some embodiments, a stent body can be formed of the radiopaque polymer. For example, body can be formed from a polyurethane radiopaque polymer with PLLA, PLGA, or PLLA or PLGA segments and elastomeric segments. A therapeutic coating can be applied over the body. Embodiments of the radiopaque polymer disclosed herein may be formed into a polymer construct, such as a tube or sheet which can be rolled or bonded to form a tube. An implantable medical device can then be formed from the construct. For example, a stent can be fabricated from a tube by laser machining a pattern in to a tube. In another embodiment, a polymer construct may be formed from the polymer using an injection molding apparatus.

As indicated above, it is important for a stent to have high radial strength so that once it is deployed from the crimped state, it can support a lumen. In general, deforming a polymer construct can strengthen the polymer of the construct along an axis of deformation. In some embodiments of fabricating a stent from a polymer tube, the polymer tube can be radially expanded to increase the radial strength of the tube. The stent can then be fabricated from the polymer tube in its expanded state. Additionally, it has been observed that radially deforming a tube prior to cutting stent patterns increases the toughness of the stent scaffolding. In particular, the radial deformation reduces or eliminates cracks and breaking of the stent struts.

In other embodiments, the radiopaque polymer disclosed herein may be used to form a coating over a device body, such as a stent body. In an embodiment, the polymer segments of P₁ and P₂ may be DL-lactide. The polyurethane PDLA radiopaque polymer may further include a therapeutic agent.

In other embodiments, the stent body can include an abluminal layer and a luminal layer with one of the layers formed from a radiopaque polyurethane. Such a stent body can be formed from a tube having an inner layer and an outer layer, such that one of the layers is composed of a radiopaque polyurethane polymer. Such a tube can be formed through coextrision. A stent body can then be formed by cutting a stent pattern in the coextruded tube.

Representative examples of polymers that may be used to fabricate an implantable medical device include, but are not limited to, poly(N-acetylglucosamine) (Chitin), Chitosan, poly(hydroxyvalerate), poly(lactide-co-glycolide), poly(hydroxybutyrate), poly(hydroxybutyrate-co-valerate), polyorthoester, polyanhydride, poly(glycolic acid), poly(glycolide), poly(L-lactic acid), poly(L-lactide), poly(D,L-lactic acid), poly(L-lactide-co-glycolide); poly(D,L-lactide), poly(caprolactone), poly(trimethylene carbonate), polyethylene amide, polyethylene acrylate, poly(glycolic acid-co-trimethylene carbonate), co-poly(ether-esters) (e.g. PEO/PLA), polyphosphazenes, biomolecules (such as fibrin, fibrinogen, cellulose, starch, collagen and hyaluronic acid), polyurethanes, silicones, polyesters, polyolefins, polyisobutylene and ethylene-alphaolefin copolymers, acrylic polymers and copolymers other than polyacrylates, vinyl halide polymers and copolymers (such as polyvinyl chloride), polyvinyl ethers (such as polyvinyl methyl ether), polyvinylidene halides (such as polyvinylidene chloride), polyacrylonitrile, polyvinyl ketones, polyvinyl aromatics (such as polystyrene), polyvinyl esters (such as polyvinyl acetate), acrylonitrile-styrene copolymers, ABS resins, polyamides (such as Nylon 66 and polycaprolactam), polycarbonates, polyoxymethylenes, polyimides, polyethers, polyurethanes, rayon, rayon-triacetate, cellulose, cellulose acetate, cellulose butyrate, cellulose acetate butyrate, cellophane, cellulose nitrate, cellulose propionate, cellulose ethers, and carboxymethyl cellulose.

Additional representative examples of polymers that may be especially well suited for use in fabricating an implantable medical device according to the methods disclosed herein include ethylene vinyl alcohol copolymer (commonly known by the generic name EVOH or by the trade name EVAL), poly(butyl methacrylate), poly(vinylidene fluoride-co-hexafluororpropene) (e.g., SOLEF 21508, available from Solvay Solexis PVDF, Thorofare, N.J.), polyvinylidene fluoride (otherwise known as KYNAR, available from ATOFINA Chemicals, Philadelphia, Pa.), ethylene-vinyl acetate copolymers, and polyethylene glycol.

For the purposes of the present invention, the following terms and definitions apply:

As indicated above, an implantable medical device such as a stent can be medicated by incorporating an active agent in a coating over the device or within the substrate of the device.

For the purposes of the present invention, the following terms and definitions apply:

“Molecular weight” can refer to the molecular weight of individual segments, blocks, or polymer chains. “Molecular weight” can also refer to weight average molecular weight or number average molecular weight of types of segments, blocks, or polymer chains. The number average molecular weight (Mn) is the common, mean, average of the molecular weights of the individual segments, blocks, or polymer chains. It is determined by measuring the molecular weight of N polymer molecules, summing the weights, and dividing by N:

$${\overline{M}}_{n} = \frac{{\sum }_{i} {N}_{i} {M}_{i}}{{\sum }_{i} {N}_{i}}$$

where Ni is the number of polymer molecules with molecular weight Mi. The weight average molecular weight is given by

$${\overline{M}}_{w} = \frac{{\sum }_{i} {N}_{i} {M}_{i}^{2}}{{\sum }_{i} {N}_{i} {M}_{i}}$$

where Ni is the number of molecules of molecular weight Mi.

The “glass transition temperature,” Tg, is the temperature at which the amorphous domains of a polymer change from a brittle vitreous state to a solid deformable or ductile state at atmospheric pressure. In other words, the Tg corresponds to the temperature where the onset of segmental motion in the chains of the polymer occurs. When an amorphous or semicrystalline polymer is exposed to an increasing temperature, the coefficient of expansion and the heat capacity of the polymer both increase as the temperature is raised, indicating increased molecular motion. As the temperature is raised the actual molecular volume in the sample remains constant, and so a higher coefficient of expansion points to an increase in free volume associated with the system and therefore increased freedom for the molecules to move. The increasing heat capacity corresponds to an increase in heat dissipation through movement. Tg of a given polymer can be dependent on the heating rate and can be influenced by the thermal history of the polymer. Furthermore, the chemical structure of the polymer heavily influences the glass transition by affecting mobility.

“Stress” refers to force per unit area, as in the force acting through a small area within a plane. Stress can be divided into components, normal and parallel to the plane, called normal stress and shear stress, respectively. True stress denotes the stress where force and area are measured at the same time. Conventional stress, as applied to tension and compression tests, is force divided by the original gauge length.

“Strength” refers to the maximum stress along an axis which a material will withstand prior to fracture. The ultimate strength is calculated from the maximum load applied during the test divided by the original cross-sectional area.

“Modulus” may be defined as the ratio of a component of stress or force per unit area applied to a material divided by the strain along an axis of applied force that results from the applied force. For example, a material has both a tensile and a compressive modulus. A material with a relatively high modulus tends to be stiff or rigid. Conversely, a material with a relatively low modulus tends to be flexible. The modulus of a material depends on the molecular composition and structure, temperature of the material, amount of deformation, and the strain rate or rate of deformation. For example, below its Tg, a polymer tends to be brittle with a high modulus. As the temperature of a polymer is increased from below to above its Tg, its modulus decreases.

“Strain” refers to the amount of elongation or compression that occurs in a material at a given stress or load.

“Elongation” may be defined as the increase in length in a material which occurs when subjected to stress. It is typically expressed as a percentage of the original length.

Elongation to Break is the strain on a sample when it breaks. It is usually is expressed as a percent.

“Toughness” is the amount of energy absorbed prior to fracture, or equivalently, the amount of work required to fracture a material. One measure of toughness is the area under a stress-strain curve from zero strain to the strain at fracture. The stress is proportional to the tensile force on the material and the strain is proportional to its length. The area under the curve then is proportional to the integral of the force over the distance the polymer stretches before breaking. This integral is the work (energy) required to break the sample. The toughness is a measure of the energy a sample can absorb before it breaks. There is a difference between toughness and strength. A material that is strong, but not tough is said to be brittle. Brittle substances are strong, but cannot deform very much before breaking.

“Solvent” is defined as a substance capable of dissolving or dispersing one or more other substances or capable of at least partially dissolving or dispersing the substance(s) to form a uniformly dispersed solution at the molecular- or ionic-size level at a selected temperature and pressure. The solvent should be capable of dissolving at least 0.1 mg of the polymer in 1 ml of the solvent, and more narrowly 0.5 mg in 1 ml at the selected temperature and pressure, for example, ambient temperature and ambient pressure.

### EXAMPLES

The examples and experimental data set forth below are for illustrative purposes only and are in no way meant to limit the invention. The following examples are given to aid in understanding the invention, but it is to be understood that the invention is not limited to the particular materials or procedures of examples. The Examples below are provided by way of illustration only and not by way of limitation. The parameters and data are not to be construed to limit the scope of the embodiments of the invention.

### Example 1

### Preparation of Radiopauqe PLLA Material

Step 1: Synthesis of PLLA Short Chains with Hydroxyl End Groups (PLLA Diol)

40 g of LLA, 3.1 g ethylene glycol and 80 ml xylene are added into a reactor free of moisture and oxygen. All chemicals are mixed through mechanical stirring and the solution is heated to 90° C. Then 140 mg stannous octoate is added into the reactor and the solution is stirred at 120° C. for 24 h to form PLLA short chains with hydroxyl groups at two ends.

Step 2: Synthesis of PLLA Long Chains with —NH Reactive Functional Groups in the Backbone

Decrease the temperature of the reaction mixture to 90° C., then add 7 g diisocyanatobutane as PLLA chain connector into the reactor and stir the polymerization solution for another 12 hours.

### Step 3: Synthesis of Radiopaque PLLA Material

12 hr later, 40 g triiodobenzoyl chloride and 5 ml of triethyl amine are added into the reactor and stirred 10 h to finish the grafting reaction.

The final product is precipitated into 500 ml methanol and dried in a vacuum oven at 80° C. until constant weight.

### Example 2

### Radiopaque PLGA Material

Step 1: Synthesis of PLGA Short Chains with Hydroxyl End Groups (PLGA Diol)

40 g of LLA, 10 g GA, 3.1 g ethylene glycol and 80 ml xylene are added into a reactor free of moisture and oxygen. All chemicals are mixed through mechanical stirring and the solution is heated to 90° C. Then 140 mg stannous octoate is added into the reactor and the solution is stirred at 120° C. for 24 h to form PLGA short chains with hydroxyl groups at two ends.

Step 2: Synthesis of PLGA Long Chains with —NH Reactive Functional Groups in the Backbone

Decrease the temperature of the reaction mixture to 90° C., then add 7 g diisocyanatobutane as PLGA chains connector into the reactor and stir the polymerization solution for another 12 hours.

### Step 3: Synthesis of Radiopaque PLGA Material

12 hr later, 40 g triiodobenzoyl chloride and 5 ml of triethyl amine are added into the reactor and stirred 10 h to finish the grafting reaction.

The final product is precipitated into 500 ml methanol and dried in a vacuum oven at 80° C. until constant weight.

### Example 3

### Preparation of Radiopaque Material Containing PLLA Hard and PCL or PDO soft blocks

Step 1: Synthesis of PLLA Diol Oligomer (Mn=2000 g/mol)

In this example, ethylene glycol (Mn 62 g/mol) as initiator, LLA as monomer, and stannous octoate as catalyst are used.

500 g LLA, 8 g ethylene glycol (0.125 mol), and 125 mg stannous octoate are added into a reactor free of moisture and oxygen. All chemicals are heated to 130° C. for 12 h to obtain PLLA diol oligomer.

Step 2: Synthesis of PDO or PCL Diol Oligomer (Mn=1000 g/mol)

In this example, ethylene glycol as initiator, DO or CL as monomer, and stannous octoate as catalyst are used.

100 g DO or CL, 3.1 g ethylene glycol (0.05 mol), and 25 mg stannous ocotate are added into a reactor free of moisture and oxygen. All chemicals are heated to 120° C. for 12 h to obtain PDO or PCL diol oligomer.

Step 3: Synthesis of Polyurethane Block Copolymer with PLLA Hard Blocks and PDO or PCL Soft Blocks

In this example, PLLA diol, PDO or PCL diol as oligomers, diisocyanatobutane (Mn=140 g/mol) as connector, stannous octoate as catalyst are used.

500 g PLLA diol, 100 g PDO or PCL diol, 48 g diisocyanatobutane (0.34 mol), 2 L anhydrous tetrahydroftiran (THF), and 250 mg stannous ocotate are added into a reactor free of moisture and oxygen. All chemicals are stirred at 60° C. for 12 h to obtain PLLA-b-PDO or PLLA-b-PCL polyurethane with PLLA as a dominant component.

Step 4: Addition of Radiopaque Groups onto Polymer Chain Containing Hard and Soft Blocks

12 hr later, 400 g triiodobenzoyl chloride and 50 ml of triethyl amine are added into the reactor and stirred 6 h to finish the grafting reaction.

The final product is precipitated into 5 L methanol and dried in a vacuum oven at 80° C. until constant weight.

### Example 4

### Preparation of Radiopaque Material from PLLA-b-PCL-b-PLLA Prepolymer

Step 1: Synthesis of PLLA-b-PCL-b-PLLA Short Chains with Hydroxyl End Groups (PLLA-b-PCL-b-PLLA diol)

100 g CL, 31 g ethylene glycol and 1 L xylene are added into a reactor free of moisture and oxygen. All chemicals are mixed through mechanical stirring and the solution is heated to 90° C. Then 1400 mg stannous octoate is added into the reactor and the solution is stirred at 120° C. for 24 h. Then 250 g of LLA is added into the reactor to react for another 48 h to form PLLA-b-PCL-b-PLLA short chains with hydroxyl groups at two ends.

Step 2: Synthesis of PLLA-b-PCL-b-PLLA Long Chains with —NH Reactive Functional Groups in the Backbone

Decrease the temperature of the reaction mixture to 90° C., then add 7 g diisocyanatobutane as PLLA-b-PCL-b-PLLA chains connector into the reactor and stir the polymerization solution for another 12 hours.

### Step 3: Synthesis of Radiopaque PLLA-b-PCL-b-PLLA Material

12 hr later, 400 g triiodobenzoyl chloride and 50 ml of triethyl amine are added into the reactor and stirred 6 h to finish the grafting reaction.

The final product is precipitated into 5 L methanol and dried in a vacuum oven at 80° C. until constant weight.

### Example 5

### Stent Preparation from Various Radiopaque Material Made in Example 1 to Example 4

Step 1: Extrude tubes with designated inside diameter (ID) and outside diameter (OD) (0.02″ and 0.07″) from various synthesized radiopaque materials prepared in Example 1 to Example 4 using a single screw extruder at 210° C.
Step 2: Expand the extruded tubes to improve their mechanical properties along the axial and radial directions.
Step 3: Cut stents from the expanded tubes using a femto second laser, crimp down to 0.05″ diameter, and sterilize all stents after crimping.

While particular embodiments of the present invention have been shown and described, it will be obvious to those skilled in the art that changes and modifications can be made without departing from this invention in its broader aspects. Therefore, the appended claims are to encompass within their scope all such changes and modifications as fall within the true spirit and scope of this invention.

What is claimed is:

1. A stent comprising a stent body fabricated from a polymer including degradable block copolymer segments joined by di-urethane linkages, the block copolymer segments comprising hard blocks and soft blocks, the soft blocks being tougher than the hard blocks at physiological conditions, and wherein radiopaque functional groups are chemically bonded to the polymer.

2. The stent of claim 1, wherein the radiopaque functional groups are chemically bonded to the di-urethane linkages along the backbone of the copolymer.

3. The stent of claim 1, wherein the radiopaque functional groups are chemically bonded to the ends of the polymer.

4. The stent of claim 1, wherein the radiopaque functional groups are derived from compounds selected from the group consisting of triiodobenzoyl chloride, triiodobenzoyl bromide and triidobenzoic acid.

5. The stent of claim 1, wherein the di-urethane linkages comprise urethane groups linked by a functional group selected from the group consisting of an aliphatic functional group, or an aromatic functional group.

6. The stent of claim 1, wherein the hard blocks are selected from the group consisting of PLLA, PGA, and PLGA.

7. The stent of claim 1, wherein the soft blocks are selected from the group consisting of PCL, PTMC, PHB, PDO, P(CL-co-GA), P(TMC-co-GA), and P(DO-co-GA).

8. The stent of claim 1, wherein the hard blocks have a Tg above body temperature and the soft blocks have a Tg below body temperature.

9. The stent of claim 1, wherein a degradation rate of the soft blocks is faster than the hard blocks, wherein the degradation of the soft blocks decreases the degradation time of the stent body.

10. The stent of claim 1, wherein the soft blocks form a dispersed elastomeric phase within a glassy polymeric matrix that includes the hard blocks.

11. A stent comprising a stent body fabricated from a polymer including degradable hard block polymer segments and degradable soft block polymer segments joined by di-urethane linkages, the soft blocks being tougher than the hard blocks at physiological conditions, and wherein radiopaque functional groups are chemically bonded to the polymer.

12. The stent of claim 11, wherein the radiopaque functional groups are chemically bonded to the di-urethane linkages along the backbone of the copolymer.

13. The stent of claim 11, wherein the radiopaque functional groups are chemically bonded to the ends of the polymer.

14. The stent of claim 11, wherein the radiopaque functional groups are derived from compounds selected from the group consisting of triiodobenzoyl chloride, triiodobenzoyl bromide, and triidobenzoic acid.

15. The stent of claim 11, wherein the di-urethane linkages comprise urethane groups linked by a functional group selected from the group consisting of an aliphatic functional group or an aromatic functional group.

16. The stent of claim 11, wherein the hard blocks are selected from the group consisting of PLLA, PGA, and PLGA.

17. The stent of claim 11, wherein the soft blocks are selected from the group consisting of PCL, PTMC, PHB, PDO, P(CL-co-GA), P(TMC-co-GA), and P(DO-co-GA).

18. The stent of claim 11, wherein the hard blocks have a Tg above body temperature and the soft blocks have a Tg below body temperature.

19. The stent of claim 11, wherein a degradation rate of the soft blocks is faster than the hard blocks, wherein the degradation of the soft blocks decreases the degradation time of the stent body.

20. The stent of claim 11, wherein the soft blocks form a dispersed elastomeric phase within a glassy polymeric matrix that includes the hard blocks.

21. A method of fabricating a stent comprising:
reacting a biodegradable block polymer diol comprising a hard block and soft block with a di-isocyanate to form a polyurethane comprising biodegradable block copolymer segments joined by di-urethane linkages;
grafting radiopaque functional groups to the backbone of the biodegradable polyurethane to form a radiopaque polymer; and
forming a stent body comprising the radiopaque polymer.

22. The method of claim 21, wherein the radiopaque groups are grafted onto the backbone through reaction of a radiopaque compound with —NH groups of the di-urethane linkages.

23. The method of claim 21, wherein the radiopaque compound is selected from the group consisting of triiodobenzoyl bromide, triiodobenzoyl chloride, and triidobenzoic acid.

24. The method of claim 21, wherein the hard block biodegradable polymer diol is selected from the group consisting of PLLA diol and PLGA diol.

25. The method of claim 21, wherein the soft block biodegradable polymer diol is selected from the group consisting of PCL diol, PTMC diol, PHB diol, and PDO diol and the soft block biodegradable polymer diol is selected from the group consisting of P(CL-co-GA) diol, P(TMC-co-GA) diol, and P(DO-co-GA) diol.

26. The method of claim 21, wherein the di-isocyanate is selected from the group consisting of di-isocyanatomethane, di-isocyanatobutane, di-isocyanatoethane, di-isocyanatohexane, di-isocyanatocubane, lysine di-isocyanate, and di-isocyanatocycloehexane.

27. A method of fabricating a stent comprising:
reacting a biodegradable hard block polymer diol, a soft block polymer diol, and a di-isocyanate to form a polyurethane comprising hard block polymer segments and soft block polymer segments joined by di-urethane linkages;
grafting radiopaque functional groups to the backbone of the biodegradable polyurethane to form a radiopaque polymer; and
forming a stent body comprising the radiopaque polymer.

28. The method of claim 27, wherein the radiopaque groups are grafted onto the backbone through reaction of a radiopaque compound with —NH groups of the di-urethane linkages.

29. The method of claim 27, wherein the radiopaque compound is selected from the group consisting of triiodobenzoyl bromide, triiodobenzoyl chloride, and triidobenzoic acid.

30. The method of claim 27, wherein the hard block polymer diol is selected from the group consisting of PLLA diol and PLGA diol and the soft block polymer diol is selected from the group consisting of PCL diol, PTMC diol, PHB diol, and PDO diol.

31. The method of claim 27, wherein the soft block biodegradable polymer diol is selected from the group consisting of P(CL-co-GA) diol, P(TMC-co-GA) diol, and P(DO-co-GA) diol.
